(19) United States
(12) Patent Application Publication
(10) Pub. No.: US 2004/0226771 A1
(43) Pub. Date: Nov. 18, 2004

(54) Stethoscope

(76) Inventor: Marc S. Werblud, Seattle, WA (US)

Correspondence Address: Christopher A. Wiklof, 3531 99th St. SE, Everett, WA, 98208, US

(21) Appl. No.: 10/439,603

(22) Filed: May 15, 2003

(51) Int. Cl. A61B 7/02

(52) U.S. Cl. 181/131

(57) ABSTRACT

A pneumatic audio system, and particularly a stethoscope, provides enhanced user comfort using ear tips that anchor to the user's ears. The anchoring of the ear tips allows lower binaural spring pressure. The stethoscope offers enhanced audio performance by using an acoustically-smooth air column that transmits sound pressure with maximum audio fidelity. Optional inner air tubes carry the air column from the chest piece to the ear tips, thus avoiding air column obstructions and material changes and enhancing audio quality. The ear tips provided enhanced audio isolation from the ambient environment. The air column is carried by a structure that provides enhanced acoustic isolation from the ambient environment. The air column is vented to optimize balance between audio isolation and audio fidelity. A replaceable transducer cell enhances acoustic isolation and sound quality. The stethoscope includes visual indicators to aid the user in readily identifying the stethoscope and its adjustment parameters.

### FIELD OF THE INVENTION

The present invention relates to pneumatically-coupled audio systems, and more particularly to stethoscopes.

### BACKGROUND OF THE INVENTION

Historically, a physician might have placed his ear against a patient's chest in an effort to detect suspicious sounds. The practice of listening to sounds emanating from a patient and drawing diagnoses or making a health assessment therefrom is called auscultation, the verb form being to auscultate. Unaided auscultation was an effective enough practice that the acoustic stethoscope was invented.

A conventional acoustic stethoscope conveys sound from inside a patient to the ears of a physician or other health-care provider via acoustic tubes (also called air tubes) that isolate the signal. The acoustic tubes are coupled to the patient's body through a bell that may optionally be covered by an acoustic diaphragm. Stethoscopes are useful in that they both spatially isolate sounds and, through the use of an enlarged bell or parabola-shaped sound collecting area, may help to amplify sounds to a level high enough to distinguish over the ambient noise. Frequently, modern acoustic stethoscopes have two bells, each having a different diameter. The larger, diaphragm-covered bell is often used for general practice while the smaller diameter bell, frequently not covered by a diaphragm, is often used to distinguish more subtle sounds such as heart sounds.

One issue faced by prior art stethoscopes is the relatively poor fit of the stethoscope's earpieces. Typically the fit is so poor as to require significant force against the user's ears. This force is typically produced by a binaural spring assembly that attaches each of the stethoscope's individual binaural eartubes to the stethoscope's flexible tubing.

Another issue faced by prior art stethoscopes is relatively poor acoustic isolation from ambient noise. The binaural spring assembly force is also required to maintain the position the ear tip lumen in general proximity of the outer ear canal opening, thus aiding in isolation from ambient noise. One might be tempted to try to gain greater isolation from ambient noise by inserting the ear tips more deeply. Unfortunately, this not only causes discomfort, but also can result in decreased stethoscope performance, especially with respect to auscultation sound volume.

Numerous attempts have been made to improve acoustic performance. For instance U.S. Pat. No. 4,055,233 describes “a self forming ear plug element for use with a stethoscope . . . A conical potion having a sound-transmitting orifice enters the ear. A thin radially extensive shallow-conical acoustic flange extends outwardly from the major diameter part of the conical portion. Being soft, the flange easily conforms to the concha portion of the ear to increase the sound pressure level of the transmitted signal and to greatly attenuate ambient noise.”

Unfortunately, such a flange only generally positions the ear tip lumen proximate to the outer ear canal opening. Because users exhibit considerable variability in the size and shape of their aural conchae, there is a corresponding variability in sealing and comfort. Furthermore, the ear tip of the '233 patent results in a binaural insertion angle that is determined by the shape of the concha, rather than enabling optimal alignment with the outer ear canal, an effect that may adversely affect audio quality. Additionally, the ear tip disclosed in the '233 patent may require considerable binaural spring tension to hold the ear tip in place.

In prior art stethoscopes, the compression of the user's outer ear canal and concha needed to keep the stethoscope in place during auscultation is often uncomfortable for the user, even after only a brief period, and can irritate the concha or outer ear canal. The combination of inwardly compressive binaural force and conventional bulb-shape stethoscope ear tip configurations applies force on user's ear canal tissues to constrict the outer ear canal opening, thereby reducing audio quality in addition to the degradation in comfort.

These shortcomings may result both in significant variability in objective audio performance and variability in subjective comfort among users.

### OVERVIEW OF THE INVENTION

The present invention describes pneumatic audio systems, of which the stethoscope is an embodiment, and components thereof, that improve user comfort and enhance audio performance, with reduced cost and enhanced manufacturability.

In another aspect, the disclosure describes a stethoscope and stethoscope components that improve user comfort. For instance, the improved ear tips of the present disclosure not only fit the user better, but improved mechanical coupling to the ear reduces the need for external pressure on the ear concha and outer ear canal by spring tension. Thus, a stethoscope with reduced binaural spring tension may be realized.

In yet another aspect, the present disclosure describes improved ear tips that are designed to fit any ear, regardless of size. This is accomplished, in part, by the fact that the ear tips comprise a series of tapering radial flanges. On individuals with smaller outer ear canals, the more distal (as viewed from the air tube), smaller diameter flanges contact the ear canals and form a tight seal. On individuals with larger outer ear canals, more proximal, larger diameter flanges contact the outer ear canals and form a seal.

In another aspect, the ear tips may be trimmed to fit. Instructions may be included with the ear tips to guide such efforts. In the particular case of the ear tips shown herein, one or two flanges may be trimmed off the distal end to achieve a better fit. Generally, the larger the outer ear canal, the more trimming is appropriate.

In still another aspect, ear tips with improved acoustic isolation are described. The improved acoustic isolation is due, in part, to the seating of at least two flanges against the inner ear canal. By such seating of two adjacent flanges, the airspace therebetween creates additional acoustic isolation.

In another aspect, a stethoscope with improved audio fidelity is disclosed. This is achieved, in part, by the use of continuous cross-section air tubes. In a preferred embodiment, inner air tubes, formed of urethane for instance, extend fully from the sound collection chamber of the chest piece (also referred to as the stethoscope head) to the user's ear canal, held in place and properly directed thereto by the ear tips. Thus, sound artifacts caused by transitions of cross-section and transitions between materials are eliminated.

The optional inner air tubes are held, guided, and directed by the stethoscope or pneumatic audio system. The system eliminates kinking or other constrictions of the air column(s), thus enhancing frequency response and transient response characteristics. In some embodiments, the inner air tubes provide a dimensionally uniform and smooth inner surface. In another embodiment, the inner air tubes are made of an acoustically reflective material. The system creates air columns that transmit sound pressure while minimizing micro-acoustic efficiency losses, thereby maximizing audio quality. The air columns are isolated from ambient noise by a combination of structures, materials, and geometry; thus improving the ability of the user to hear subtle tonal, amplitude, and transient characteristics in the audio source. Many of the characteristics are user-selectable or user-adjustable.

In another aspect, a chest piece with inner air tubes is disclosed. In some embodiments, the inner air tubes are cut at differing lengths to increase the sensation of stereo separation. In other embodiments, inner air tubes are cut on a bias to improve audio volume and pass-band width. In other embodiments, inner air tubes are straight cut on a bias at a length to contact the diaphragm. This arrangement is especially advantageous for transmitting lower frequency signals. In still other embodiments, inner air tubes are cut on a bias with a serrated edge, the serration tips contacting the diaphragm. Contacting serrated tips are advantageous for transmitting low frequency signals while extending upper frequency response as well.

Another aspect that improves audio fidelity is the axial and unobstructed alignment of the ear tip lumen to the user's outer ear canal. Whereas prior art stethoscopes were held in place by considerable spring tension, one aspect of the present invention is to decrease binaural spring tension, and hence compressive spring force on the conchae, thus decreasing the force and attendant aural constriction of the outer ear canal. The combination of improved alignment of the ear tip passage with the outer ear canal, reduction of induced constriction of the outer ear canal, and maintenance of the ear tip air passage cross-sectional shape and volume all contribute to improved audio quality.

In another aspect, the ear tips are convertible, also working with lower cost stethoscopes that don't include inner air tubes.

To bring inner air tubes fully to the chest piece sound collection chamber, a transition fitting is disclosed.

The inventor has discovered that maximization of audio quality requires venting of the air tubes. It is believed that such venting allows the sound pressure waves to travel without impediment. Venting at the ear tips may be accomplished by trimming one or more of the ear tip flanges to allow some air passage. This venting may be performed by the user, optionally with the aid of instructions, or alternatively may be created during manufacture of the ear tips.

According to the design of the ear tips, acoustic isolation is maintained even when vented. Acoustic isolation is maintained, in part, by the airspace between flanges and by sound absorption by the ear tip materials.

In an alternative embodiment, one or more vent orifices may be formed in the ear tips.

In another alternative embodiment, adjustable venting of the ear tips allows optimization for the use environment and user preferences. Greater venting may be desired, for instance, in quieter ambient environments when sound quality is of paramount importance. Reduced venting may be desired in noisier ambient environments where noise isolation is more important than maximization of sound quality.

In another aspect, venting of the air tubes may also be present at the chest piece. In some embodiments, the chest piece includes adjustable venting that may be optimized for the use environment and user preferences. In an alternative embodiment, chest piece vents may comprise features for absorbing unwanted ambient noise, while allowing passage of air. To some extent, this may be accomplished by proper selection of material. In some embodiments, this may be accomplished by increasing the effective audio impedance through use of roughened passages, labyrinthine airflow, and/or passage through anechoic chambers.

In another aspect, air tube coupling is made at two or more positions within the sound-collection region so as to broaden the frequency range of collected sounds. In some embodiments, a mixing chamber mixes sounds of differing frequency distributions to equally distribute frequencies to both of the user's ears. In another embodiments, acoustic coupling to the two or more positions is selectable to adjust frequency distribution.

In another aspect, a sealed, replaceable transducer assembly fits within the chest piece body. In addition to providing the convenience of a replaceable transducer assembly, this arrangement provides enhanced isolation from ambient noise and may resonate at selected frequencies to enhance auscultation of sounds having corresponding frequencies.

In another aspect, a stethoscope is made easier to use by making an individually sized and adapted device easier to identify, retrieve, and use. Variable markings or coloration on the ear tips, binaurals, binaural spring, sheath, and/or chest piece for instance are used to identify an individual's stethoscope or a specific size of stethoscope. Markings or coloration may also indicate use directions, for instance which side of the binaural spring should face away from the user's chest; or audio characteristics, for instance a stethoscope optimized for noisy ambient environments vs. one optimized for high acuity auscultation.

In another aspect, a stethoscope is made to be user-adjustable with respect to fit, comfort, and/or audio characteristics.

In another aspect, user adjustments may be made visibly and/or tactilely apparent to the user by controls that include indicator features. For instance, venting controls indicate whether venting is open or closed, binaurals indicate rotation angle and head angle, the binaural spring indicates ear tip pressure, and the chest piece indicates frequency mix.

### BRIEF DESCRIPTION OF THE DRAWINGS

FIG. 1 is an enlarged isometric view of an ear tip of the present invention.

FIG. 2 is a side view of the ear tip.

FIG. 3 is an end view of the ear tip, shown from the perspective of the tip (proximal end).

FIG. 4 is an end view of the ear tip, show from the perspective of the end where the air tube enters (distal end).

FIG. 5a is an isometric view of a stethoscope of the present invention.

FIG. 5b is an isometric view of an alternative stethoscope having two chest pieces.

FIG. 6 is a side view of an ear tip mounted on the stethoscope.

FIG. 7 is a sectional side view of the ear tip mounted on the stethoscope.

FIG. 8 is a side view of an alternative ear tip design having vents.

FIG. 9a is a sectional side view of an alternative ear tip having vents.

FIG. 9b is a sectional side view of an alternative ear tip having an alternative vent design.

FIG. 10 is an isometric view of an ear tip that has been trimmed to fit.

FIG. 11 is a side view of an ear tip that has been trimmed to fit.

FIG. 12 is an isometric view of a stethoscope having an alternative chest piece design.

FIG. 13 is an isometric view of a stethoscope having a second alternative chest piece design.

FIG. 14 is a detail isometric and partial cut-away view of the mating of the binaural spring, lower sheath, and binaural with an optional inner air tube passing therethrough.

FIG. 15 is an exploded view of the fitting that connects the lower sheath and chest piece with provision for passing inner air tubes therethrough.

FIG. 16 is an isometric view of an embodiment of the proximal piece of the fitting of FIG. 15.

FIG. 17 is a side view of the fitting piece of FIG. 16.

FIG. 18 is a cross-sectional view of the fitting piece of FIGS. 16 and 17.

FIG. 19 is a cross-sectional view of another location on the fitting piece of FIGS. 16 and 17.

FIG. 20 is a side sectional view of one embodiment of the fitting piece of FIG. 16.

FIG. 21a is a cross-sectional view of the fitting piece of FIG. 20.

FIG. 22 is a side sectional view of the fitting of FIG. 16 with optional inner air tubes passing therethrough.

FIG. 23 is a cross-sectional view of the fitting piece of FIG. 22.

FIG. 24 is an isometric view of a chest piece.

FIG. 25 is a top view of the chest piece of FIG. 24.

FIG. 26 is a side view of a chest piece having inner air tubes that collect sound directly from the sound collection chamber.

FIG. 27 is a rear view of the chest piece of FIG. 26.

FIG. 28 is a top view of a chest piece having selectable venting.

FIG. 29 is a side view of the chest piece of FIG. 28.

FIG. 30 is a rear view of the chest piece of FIGS. 28 and 29.

FIG. 31 is an isometric view of a chest piece having an alternative venting design.

FIG. 32 is a side view of a chest piece having bias-cut inner air tubes.

FIG. 33 is a rear view of a chest piece having bias-cut inner air tubes.

FIG. 34 is a sectional side view of a chest piece having straight bias-cut inner air tubes contacting the diaphragm.

FIG. 35 is a sectional side view of a chest piece having serrated bias-cut inner air tubes contacting the diaphragm.

FIG. 36 is an isometric view of a binaural transducer cell.

FIG. 37 is a side view of the binaural transducer cell of FIG. 36.

FIG. 38 is an isometric exploded view of the binaural transducer cell of FIGS. 36 and 37.

FIG. 39 is an isometric view of a monaural transducer cell.

FIG. 40 is a cross-sectional view of a transducer cell mounted in a chest piece.

FIG. 41 is a side view of a transducer fitted to the alternative chest piece body of FIG. 12.

FIG. 42 is a rear view of the chest piece assembly of FIG. 41.

FIG. 43 is a top view of the chest piece assembly of FIGS. 41 and 42.

FIG. 44 is an isometric view of the chest piece assembly of FIGS. 41 through 43.

### DETAILED DESCRIPTION OF THE INVENTION

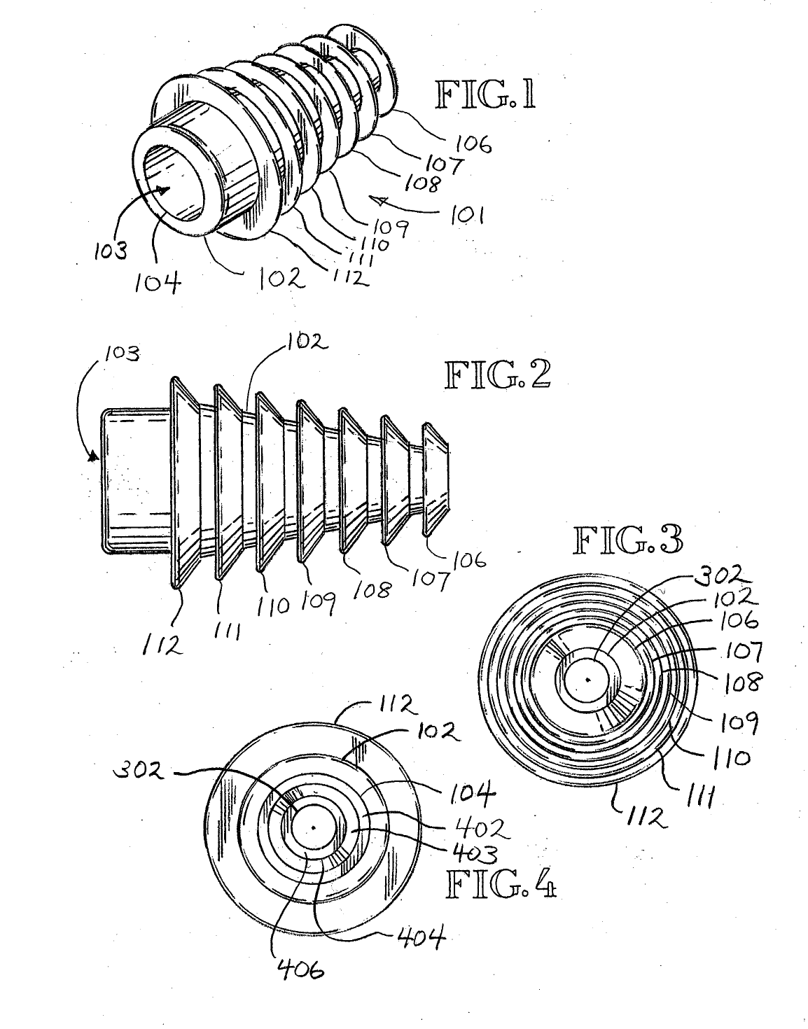

As indicated above, certain attributes of the present invention relate to an improved ear tip with enhanced manufacturability, usability, comfort, and performance. Referring to attached FIGS. 1-4, the earpiece, generally referred to by reference number 101, is comprised of a body 102 with air passage 103 formed therethrough. The body is of a generally tapered cylindrical shape with the air passage formed along its central axis.

Flanges 106-112 are formed at intervals along the length of the earpiece body, protruding from the outer circumference thereof. Preferably, flanges 106-112 are formed with progressively increasing diameter. The earpiece may be formed of a soft, elastic, non-irritating material such as rubber, silicone rubber or urethane, for instance. In a preferred embodiment, the outer flange array is comprised of a sound-attenuating polymer such as silicone, for instance, to occlude ambient infiltration.

By adjusting the geometry of the earpiece body and flanges to provide greater compliance, the earpiece may alternatively be formed from somewhat harder materials, such as nylon for instance, while maintaining reasonable comfort. This may be done, for instance, by making the flanges thinner in cross-section.

One notable aspect of the earpiece is that the flanges simultaneously reduce pressure against the outer canal of the ear and improve seating within the outer canal. Comfort is accomplished by choosing the materials and flange geometry to be compliant, deflecting when pushed against the ear. This deflection upon insertion also tends to form an anchor that partially resists removal by requiring an “over-center” motion when pulled. This “self-anchoring” capability allows the earpiece to remain in place with reduced inward pressure, thus allowing lower inward spring force than prior art ear tips, or even no inward pressure, thus improving user comfort.

Air passage 103 has several features that may be understood by reference to FIGS. 1, 3, 4, and 7. Binaural bore 104 is formed at an inner diameter substantially equal to the outer diameter of the binaurals 514. Binaural stop 402 forms a surface upon which the end of the binaurals fit. Taper 403 forms a transition between the inner diameter of the binaural and the diameter of the inner tube bore 404. In the case of a stethoscope or other pneumatic audio system that has inner air tubes 506 (also referred to as inner transmission lumens), taper 403 eases threading of the inner air tubes into the earpiece. In the case of a system that does not have inner air tubes, taper 403 improves acoustic performance by forming a smooth transition for reducing diameter which minimizes sound reflections and audio degradation. Inner air tube bore 404 is formed at an inner diameter substantially equal to the outer diameter of inner air tubes 506. It serves to hold the inner air tubes securely in a manner that provides both structural integrity, reducing the tendency of the inner air tube to be crushed, and in proper alignment with the user's outer ear canal, for maximization of audio performance.

Inner air tube stop 406 forms a surface upon which the end of the inner air tubes 506 fit. Exit passage 302 is the final passage through which sound waves travel to enter the user's outer ear canal. It is formed at substantially the same inner diameter as the inner diameter of inner air tubes 506, thus eliminating undue air column diameter changes, eliminating obstructing features, and thereby maximizing audio quality. The smooth transitions of the ear tip also reduce turbulence, directional changes, and other undesirable acoustic effects when used without the optional inner air tubes.

Exit passage 302 may be formed to have a smooth inner surface for minimization of audio artifacts. In a preferred embodiment, ear tip 101 is formed in a geometry that has similar air passage characteristics as those of the inner air tubes. This geometry provides improved marginal connectivity of ear canal and exit passage 302, thus providing a smooth transition from the ear tip to the user's outer ear canal. In this case, sound pressure waves may travel from the inner air tube 506 and out through exit passage 302 in an unimpeded manner, thereby improving audio quality.

In another preferred embodiment the inner transmission lumen is composed of a sound-reflective material such as Urethane carry the target auscultation with minimal sound pressure loss. In another embodiment, inner surfaces of inner air tubes 506 may be smooth. In another embodiment, inner surfaces of inner air tubes and ear tips may include boundary layer adhesion features, such as small longitudinal grooves for instance, to reduce the incidence of turbulence. To make a stethoscope of the present invention more adjustable for user preferences, inner air tubes of various materials, geometries, and surface characteristics may be offered to aid the user in customizing the device to his or her needs or preferences.

Ear tip 101 provides an adjustable insertion angle for optimal user comfort and sound quality. The ear tip is accurately self-positioning for optimal alignment with the user's outer ear canal. The user-determined outer sizing of the stethoscope ear tip lumen ensures custom tailoring for fit and comfort without repositioning or reducing the sizing of the inner lumen. This maintains constant performance among various dimensional and morphologic aural populations with a comfortable fit and a secure seal that is virtually independent of binaural spring assembly tension, expressed as inward pressure on the ear.

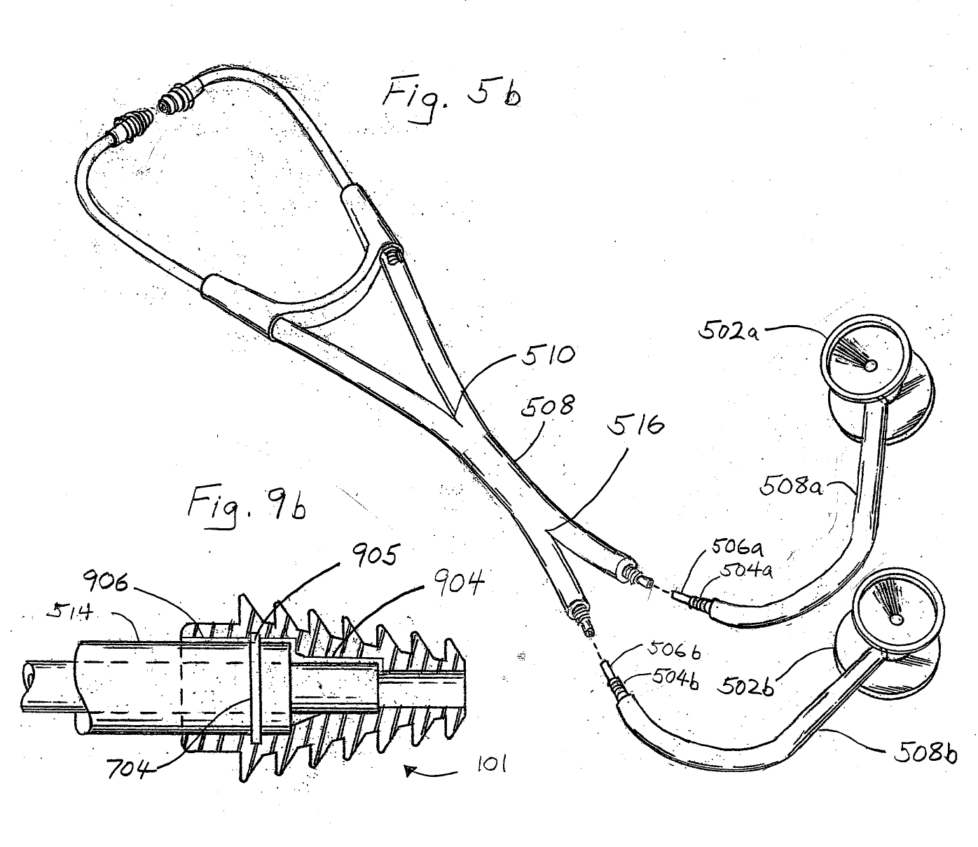

Another feature of ear tips 101 may be seen in FIG. 7. Retention groove 702 is formed within binaural bore 104 to accept retention ring 704, which in turn is formed on the outside diameter of binaural 514. Retention groove 702 and retention ring 704 mechanically couple to form an ear tip retention system that securely holds ear tip 101 on the end of binaural 514. This retention system can be especially important for withdrawal of the ear tip from the user's outer ear canal, particularly in light of the enhanced anchoring properties of the ear tip. By making the ear tip from an elastic material such as silicone rubber, the ear tip may also be easily removable from the binaural by “peeling” from the distal (binaural) end. The combination of secure attachment in use with removability when desired is an advantage of the disclosed ear tip.

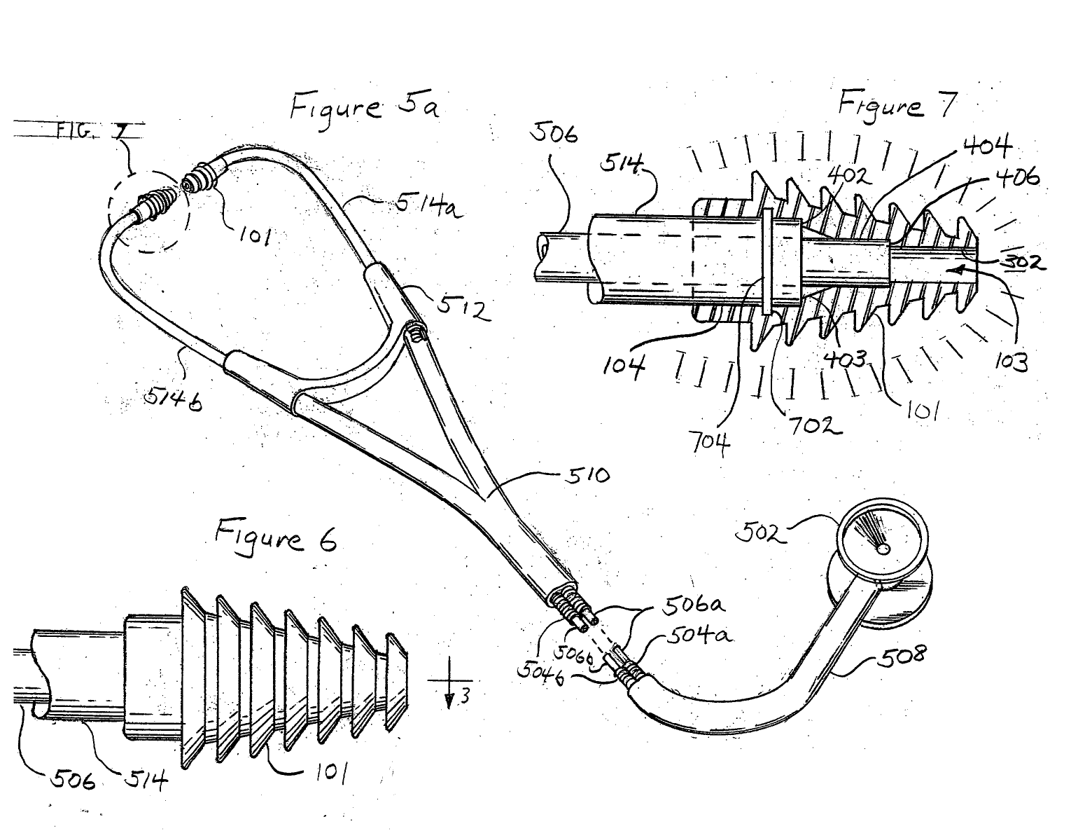

FIG. 5a illustrates a stethoscope. Chest piece 502 is formed to capture and transmit sound waves into air tubes 504a and 504b. Air tubes 504 may be formed from a tightly wound metal spring, for instance a stainless steel spring. When equipped with optional inner air tubes, air tubes 504 may be called outer air tubes. Optional inner air tubes 506a and 506b are held inside outer air tubes 504a and 504b, respectively, to present a more uniform bore in which the air column and sound waves are carried. Inner air tubes may be formed from an elastic material such as urethane, silicone, or fluorinated hydrocarbon (e.g. Teflon), for instance. Air tubes 504 and inner air tubes 506 are held within outer sheath 508, which splits at bifurcation 510 to lead the air tubes and inner air tubes to binaurals 514a and 514b. The portion of the outer sheath 508 above or proximal to bifurcation 510 may be called the upper sheath. Conversely, the portion of outer sheath 508 below or distal to bifurcation 510 may be called the lower sheath.

Binaural spring 512 performs several functions known to the art including urging ear tips 101, mounted on the end of binaurals 514 together. While prior art stethoscopes often required quite high spring pressure to hold prior art ear tips in the users ears, the self-anchoring characteristics of the present ear tips, previously described, allow a reduced spring pressure. The resultant lower pressure against the user's ears results in enhanced comfort.

FIG. 5b illustrates an alternative stethoscope having two chest pieces. In this case, sheath 508 is bifurcated both at upper bifurcation 510 and at lower bifurcation 516 to separate the air tubes. Alternatively, sheath portions 508a and 508b may be kept separate their entire lengths or externally tied together. Lower sheath 508a, outer air tube 504a, and optional inner air tube 506a couple to first chest piece 502a. Similarly, lower sheath 508b, outer air tube 504b, and optional inner air tube 506b couple to second chest piece 502b. A two-chest piece stethoscope such as is illustrated by FIG. 5b can be especially useful for application where stereo separation of noise sources can aid in auscultation.

The air tube structure of FIGS. 5a and 5b enhances isolation from ambient noise. The combination of an outer sheath 508, outer air tubes 504, and inner air tubes 506 help to isolate the air column from the ambient environment. In the case where the outer air tubes 504 are implemented as a spring coil, it is believed this structure helps to dissipate external sound as heat energy.

To ensure air tubes are directed along the centerline of the outer ear canal when the stethoscope is in use, binaurals 514a and 514b are generally rotated slightly to point the ear tips slightly forward. In some embodiments, binaural spring 512 may be marked to indicate the orientation of the stethoscope. For instance, the front or top of spring 512 may include printing, molded features, or color to indicate its forward orientation during use.

In some embodiments, binaurals 514a and 514b are rotationally adjustable to better adapt to the outer ear canal angle of specific users. In a preferred embodiment of the present invention, alignment marks, color patterns, or occluding fittings may indicate the rotation angle of the binaurals relative to the binaural springs, thus improving the ability of the user to rapidly adjust the binaurals and/or identify whether a stethoscope has binaurals that have been properly adjusted.

FIG. 6 shows a detailed side view of ear tip 101 mounted on binaural 514.

As described earlier, FIG. 7 shows a sectional side view of ear tip 101 mounted on binaural 514. Also as noted, it further shows the relationship of inner air tube 506 to features within air passage 103.

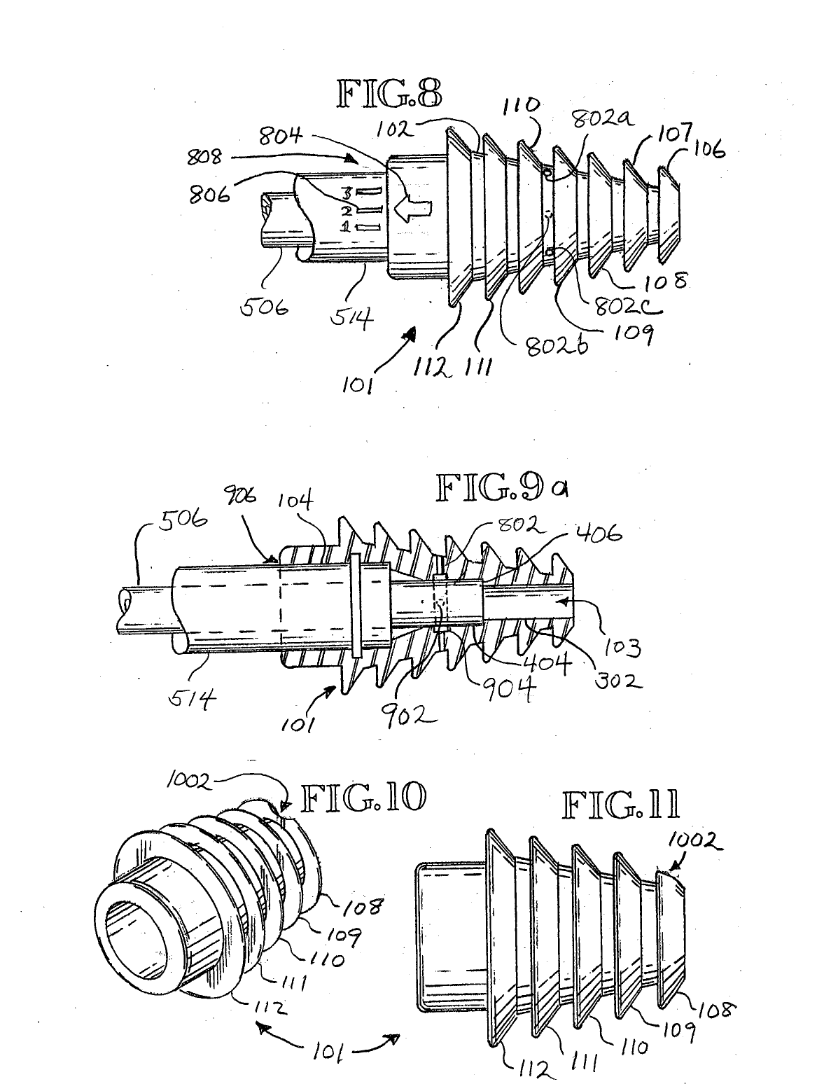

The inventor has discovered that sound quality delivered by pneumatic audio systems, and particularly by stethoscopes, is influenced by venting of the air columns. In general, more venting may be desirable in quiet ambient environments where the additional venting may improve audio quality. In contrast, less venting may be desirable in noisy environments where audio isolation is more important than audio fidelity. FIGS. 8, 9a, and 9b illustrate ear tip embodiments that provide such venting.

Referring to FIGS. 8 and 9a, alternative ear tip 101 is mounted on the end of binaural 514 as previously described. Vent passages 802, shown by example as individual passages 802a, 802b, and 802c in FIG. 8, perforate ear tip body 102. Vent passages 802 are formed so as to allow airflow between air passage 103 and the outside. In a preferred embodiment, vent passages 802 emerge from ear tip body 102 between flanges. In the example of FIGS. 8 and 9a, vent passages 802 emerge between the 4^{th} flange 109 and the 5^{th} flange 110, although in principle vent passages 802 could emerge anywhere else from alternative ear tip 101.

Orifices 902, formed in inner air tube 506, provide for communication of air pressure from the air column to plenum 904, formed as a groove within alternative ear tip 101. Vent passages 802, in turn, provide for communication of air pressure from plenum 904 to the atmosphere. Although orifice 902 is shown as a single circular hole, multiple orifices may be preferred. Additionally, the orifice(s) need not be circular; with grooves, notches, or other perforations being some example alternatives.

FIG. 9b shows an alternative embodiment of a vented ear tip. In this example, inner air tube 506 need not be perforated at all. For example, plenum 904 may be formed to run longitudinally along the inner air tube bore 404, emerging as a lateral groove in inner air tube stop 406 and/or exit passage 302, and running around the end of binaural 514 to retaining ring 704. A notch 905 in retaining ring 704 connects plenum 904 to vent passage 906, which completes communication with the atmosphere.

Vented ear tip 101 of FIGS. 8, 9a, and 9b may optionally have selectable or adjustable venting. In the example of FIGS. 8 and 9a, ear tip 101 may be rotated on binaural 514 to select a desired amount of venting. This may be accomplished, for instance, by forming plenum 404 in an arc that extends only part way around bore 404. In this way, ear tip 101 may be rotated to expose no vent holes 902, a single vent hole 902, two vent holes 902, or more than two vent holes 902, each step providing successively more venting. In the example of the embodiment of FIG. 9b, ear tip 101 may be rotated to select the amount of constriction posed by notch 905. When rotated to cause misalignment between ear tip passages 904 and 906 and notch 905, the ear tip is not vented via the vent channels. When rotated to align, passages 904, 906, and notch 905 form a variable valve that can, by the degree of alignment, determine a variable amount of venting.

Vented ear tip 101 may further include an indicator 808 to aid the user in rapidly determining the amount of venting selected. In this example, a pointer 804 molded or otherwise formed on ear tip 101 points to a scale 806 formed on binaural 514. In this example, pointer 804 and scale 806 collectively form indicator 808. Thus, as ear tip 101 is rotated to select more or less venting, indicator 808 shows the amount of venting selected.

One aspect of the ear tip of the present invention is that it may be trimmed to optimize fit to the individual. Individuals with small outer ear canals may leave the ear tip at the original length. Individuals with larger outer ear canals may customize fit by trimming off one or two flanges, for example. This may be done by cutting off the end of the earpiece with a pair of scissors or a utility knife. The tapered shape of the earpiece body, an more particularly the tapered outer diameter of the flanges, means that trimming off the end results in a larger outer diameter, thus fitting the earpiece to a larger outer ear canal. In this way, individuals with smaller outer ear canals can comfortably use the ear tip with its distal (binaural) end protruding relatively far from their outer ear canal. In contrast, individuals with larger outer ear canals who have trimmed some length off the proximal (user) end would comfortably use the ear tip with its distal (binaural) end protruding relatively less from their outer ear canal. In each case, the users would benefit from the enhanced anchoring, alignment, inner air tube support, ambient isolation, and other characteristics of the ear tips. Generally, users can expect to have at least two flanges in contact with their outer ear canals.

FIGS. 10 and 11 illustrate an ear tip 101 that has been trimmed to fit an individual with larger outer ear canals, FIG. 10 being an isometric view and FIG. 11 being a side view. It can be seen that the ear tip of FIGS. 10 and 11 retains only the five largest flanges 108-112, the two smaller flanges 106 and 107 having been trimmed off.

Another aspect of ear tip 101 is that it may be trimmed to allow venting or additional venting. For example, FIGS. 10 and 11 show trimmed area 1002 in flange 108. Trimmed area 1002 is formed to allow venting around the periphery of flange 108, thereby allowing communication of air pressure between the air column and the atmosphere along the user's outer ear canal and concha.

With the custom fit available with the ear tip of the present invention, it may be especially desirable by users to be able to rapidly distinguish a stethoscope fitted to them from other stethoscopes having different fit characteristics. A convenient way to do this is provide ear tips in a plurality of colors or color patterns. To make ear tips especially easy to distinguish, the ear tips may be produced in a plurality of bright colors, even fluorescent colors.

As an alternative to producing common ear tips in a plurality of colors, it may be desirable to produce pre-fitted ear tips, each size being produced in a particular color. Thus an enterprise that provides stethoscopes for users, for instance a hospital, could enable even new users to rapidly select the stethoscope best fitted to them.

The use of fluorescent materials could further aid users in locating and using a stethoscope even in low or no ambient light.

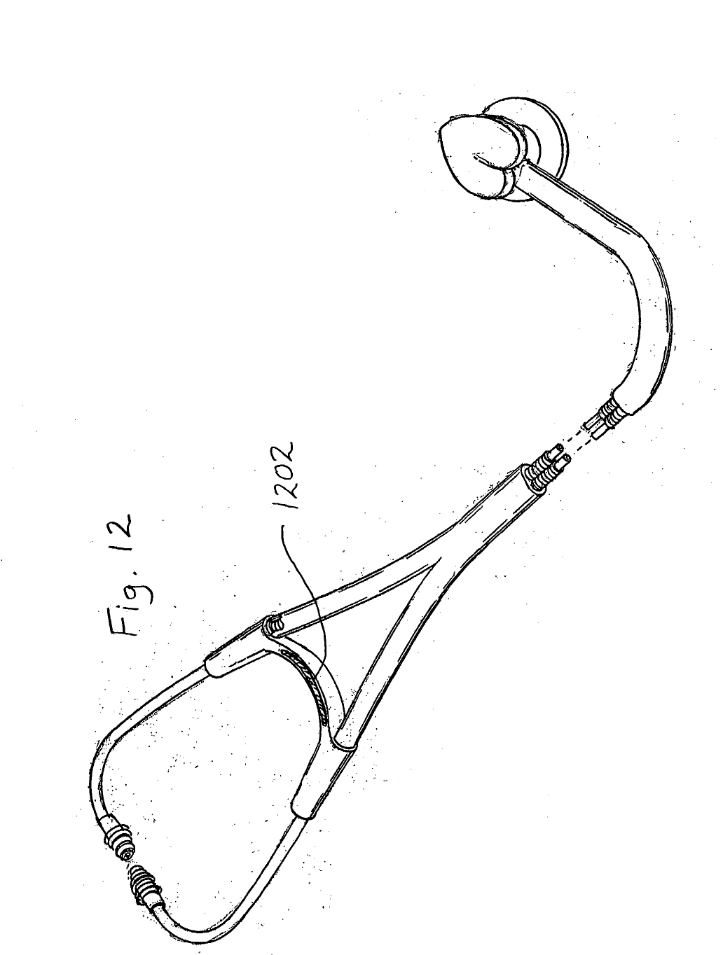

FIG. 12 is an isometric view of a stethoscope having an alternative chest piece design. FIG. 12 further illustrates color patch 1202, an alternative feature for quickly identifying the front of the stethoscope. This feature can help the practitioner quickly identify which binaural corresponds to which ear and can potentially save precious seconds during emergency situations, for instance.

FIG. 13 is an isometric view of a stethoscope having another alternative chest piece design.

FIG. 14 is an isometric view of a stethoscope showing detail where the left end of binaural spring 512 is anchored to a lower sheath and a binaural with the air column passing therethrough. The outer sheath 508 and outer air tube 504a is shown slid down the inner air tube 506a away from the binaural spring to expose features for anchoring the outer air tube to the binaural spring. Nipple 1402 anchors the outer sheath assembly to binaural spring 512. During assembly, outer sheath 508 is jammed over fitting 1402 to form a secure fit while outer air tube 504a ends at the end of nipple 1402 to avoid air column constriction.

FIG. 14 also shows an optional inner air tube 506a passing through the assembly. Inner air tube 506a is easily fed through after the assembly comprising outer sheath 508 and outer air tube 504a, binaural spring 512, and binaural 514a is fitted together. When equipped with an inner air tube, a stethoscope constructed per FIG. 14 typically exhibits improved audio performance owing to the lack of air column obstructions, materials changes, or surface changes when passing from the outer airtube 504a to binaural 514a. The stethoscope of FIG. 14 is also operative without inner airtube 506a, an embodiment that may be advantageous for reduced cost.

FIG. 14 further shows directional indicator 1404a and alternative directional indicator 1404b. Because the end of binaural spring 512 shown corresponds to the binaural to be inserted into the user's left ear, a molded “L” such as 1404a may be used to indicate this orientation to the user. Alternatively, the front of the binaural spring 512 may be indicated by appropriate molded lettering, shown by 1404b. Alternatives to molded lettering 1404a and 1404b include pad- or screen-printed lettering or symbology, or color indicators such as a fluorescent or other color patch 1202 on the front of binaural spring 512, as shown in FIG. 12.

FIG. 14 further shows a portion of binaural adjustment indicator 1406. In the example of FIG. 14, the arrow 1406 is formed on the front of binaural 514a by etching, pad printing, or other means. Arrow 1406 points to a series of rotation angle indicators 1408 on the top of binaural spring 512. Rotation angle indicators 1408 are shown as hidden lines formed peripherally around the surface where binaural 514a enters binaural spring 512. Binaural adjustment indicator 1406 and binaural rotation angle indicators 1408 collectively form a binaural indicator that aids the user in quickly ascertaining the rotation angle at which the binaural is set. The binaural rotation angle adjustment is useful for aiding users in selecting the fore-and-aft angle at which the binaurals enter the outer ear canal. In addition to aiding in maximizing comfort, this adjustment further affects sound quality in that the user may best align the ear tip air passage 103 with the outer ear canal. Superior alignment of the ear tip air passage with the outer ear canal reduces soundwave obstructions and thus improves audio characteristics.

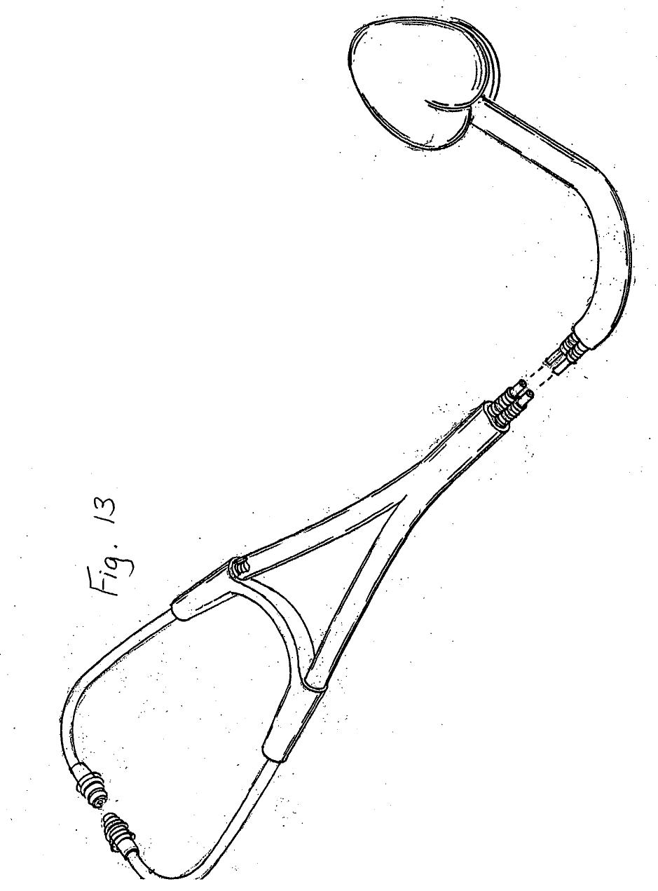

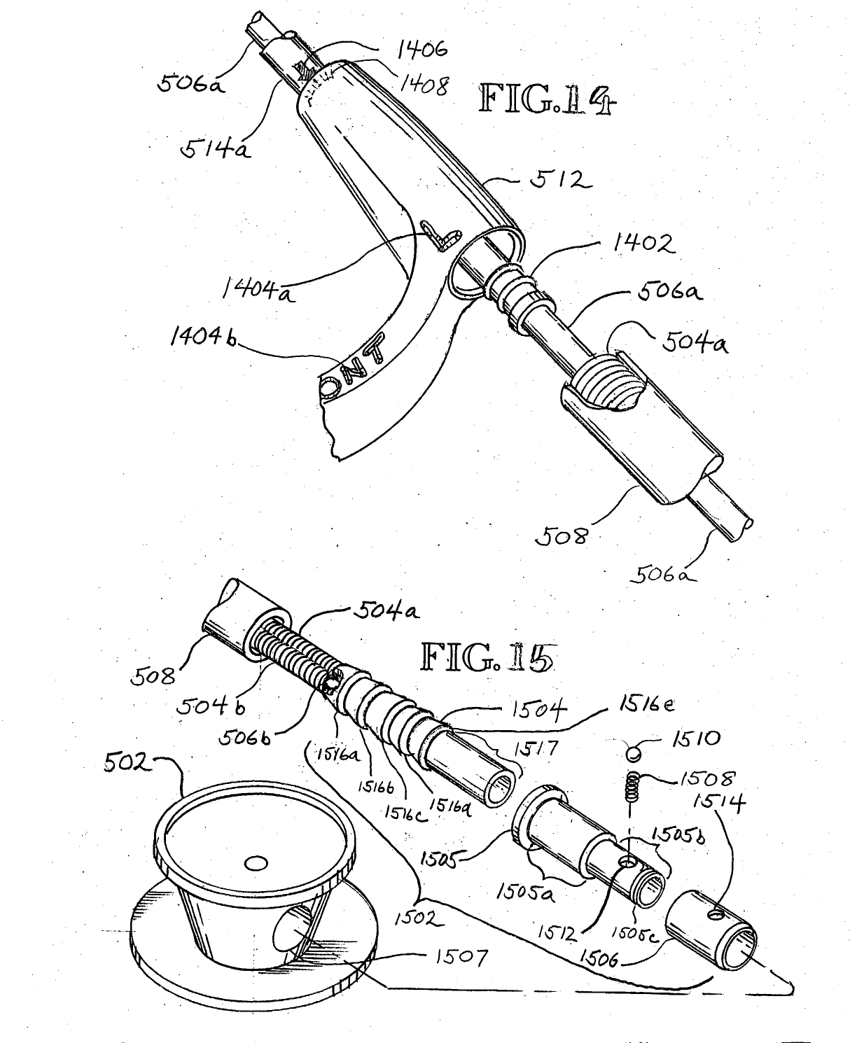

FIG. 15 is an exploded view of the fitting 1502 that connects the lower sheath 508 and outer air tubes 504a and 504b to chest piece 502. Fitting 1502 includes provision for passing inner air tubes 506a and 506b therethrough. Fitting 1502 comprises nipple 1504, middle piece 1505 and lower piece 1506.

In one embodiment, flanges 1516a through 1516e formed on fitting piece 1504 anchor lower sheath 508. In this case, fitting piece 1505 may be attached to fitting piece 1504 by press-fit, gluing, screw threads, or other mechanical fastening means. Alternatively, lower sheath 508 may be passed beyond flanges 1516a-1516e 1516e and over smooth section 1517 of fitting piece 1504. In this case, the inner diameter of section 1505a may be made somewhat larger, thus permitting fitting piece 1505 to be forced snugly over smooth section 1517, trapping sheath 508 therebetween. In this embodiment, fitting piece 1505 further secures the attachment of outer flanges 1516a-1516e to the inside of sheath 508.

Middle piece 1505 and lower piece 1506 are preferably rotatably coupled to allow the user to change the orientation of chest piece 502 relative to the rest of the stethoscope. One means for achieving this is illustrated in FIG. 15. Spring 1508 tends to force ball bearing 1510 away from spring seat 1512. When middle piece 1505 is inserted into lower piece 1506, the spring action pushes ball bearing 1510 hard against the inside of lower piece 1506. One or more holes 1514 may be formed in lower piece 1506, such holes being positioned to accept ball bearing 1510 and thus forming one or more detents.

Middle piece may be held within lower piece 1506 by an external lock ring (not shown) for instance inserted around radial groove 1505c, thus providing tensile strength. Alternatively or in addition, a radial groove (not shown) may be formed within lower piece 1506 for accepting ball bearing 1510. In this instance, the pressing of ball bearing 1510 into the radial groove adds tensile strength.

Lower piece 1506 is securely fitted to chest piece 502 via chest piece passage 1507. Lower piece 1506 may be mechanically coupled to chest piece 502 by screw threads, gluing, press-fit, welding, or other mechanical fastening means, for instance.

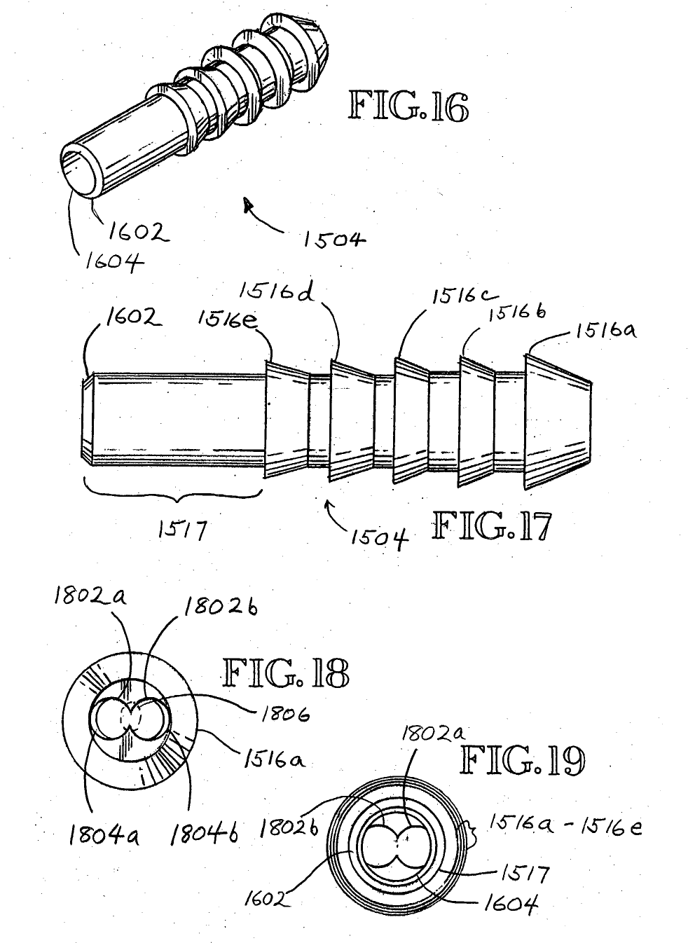

FIGS. 16, 17, 18, and 19 are detailed views of upper piece 1504 of fitting 1502. In addition to features described above, FIGS. 16 and 17 show chamfer 1602 formed on the end of smooth section 1517. FIG. 16 further draws attention to inner wall 1604, which forms the wall of a single air passage. In a preferred embodiment, the diameter of inner wall 1604 of fitting piece 1504 is made substantially equal to the inner diameter of section 1505b of piece 1505, thus creating a smooth passage for air and sound flow as well as a smooth passage to ease threading of optional inner air tubes 506a and 506b.

FIG. 18 shows the proximal end of fitting piece 1504, corresponding to the end having flanges 1516a through 1516e. In this view are shown air passages 1802a and 1802b in wall 1806. Narrowing 1802 may be formed as an overlap of the outer diameters of air passages 1802a or 1802b, and thus exhibited as opposed ridges, as shown in FIGS. 18 or 19. Alternatively, air passages 1802a and 1802b may be formed slightly apart, thus forming a septum wall 1802. In another alternative embodiment, bores 1802a and 1802b may be formed at a slightly converging angle, for instance 5 degrees, thus creating a septum 1802 that is a longitudinal wall for part of the length of fitting piece 1504, for instance along a distance approximately corresponding to flanges 1516a-1516e, and a pair of opposing ridges 1802 for another portion of the length of fitting piece 1504, for instance along a distance approximately corresponding to smooth section 1517. Surfaces 1804a and 1804b form a transition from air passages 1802a and 1802b to inner diameter 1604. They may be formed at a 10° angle to air passage 1604, for instance, using an 80° cone end mill.

In a preferred embodiment, wall 1806 is formed as close as possible to the distal end of nipple 1504 to reduce the air volume held within bore 1604.

FIG. 20 is a side sectional view of the upper fitting piece 1504 of FIGS. 16-19 and FIG. 21 is a cross-section of location 21 in FIG. 20. FIG. 20 depicts the use of fitting 1504 with inner air tubes 506a and 506b that end at transition surfaces 1804a and 1804b , respectively. Also shown in FIGS. 20 and 21 is an embodiment using a single lower outer air tube 2002 through which inner air tubes 506a and 506b pass. In this example lower outer air tube 2002 is comprised of a tightly coiled spring. In some embodiments, outer air tubes 504a and 504b are wrapped by the upper end of lower outer air tube 2002 at the sheath bifurcation 510 shown in FIG. 5. Outer air tubes 504a and 504b there separate, leading to opposite ends of binaural spring 512, being fastened thereto as shown in FIG. 14.

FIG. 22 is an alternative embodiment of FIG. 20, wherein inner air tubes 506a and 506b are threaded through the entire length of fitting piece 1504. FIG. 23 is a cross-section taken at location 23 in FIG. 22. FIG. 23 shows fitting piece 1504, shown at smooth section 1517, with inner air tubes 506a and 506b passing through. In the embodiment shown, inner air tubes 506a and 506b are elastically compressed slightly to become oval-shaped as they press against one another and against inner wall 1604.

Fitting 1502 provides a transition from the sound collection chamber to air columns that minimizes unwanted sound reflections while maximizing sound pressure transmission, transient response, and overall audio quality. When equipped with optional inner air tubes, air passages 1802a and 1802b form a seal around the inner air tubes, thus forcing substantially all collected sound pressure through the inner air tubes. When not equipped with the optional inner air tubes, air passages 1802a and 1802b are formed to direct sound pressure upward and through outer air tubes with minimal obstruction or other unwanted side effects.

As an alternative to fitting 1502, portions such as air passages 1802a and 18024b, transition surfaces 1804a and 1804b, wall 1806, and flanges 1516a through 1516c may be formed integrally with the air passage of a chest piece.

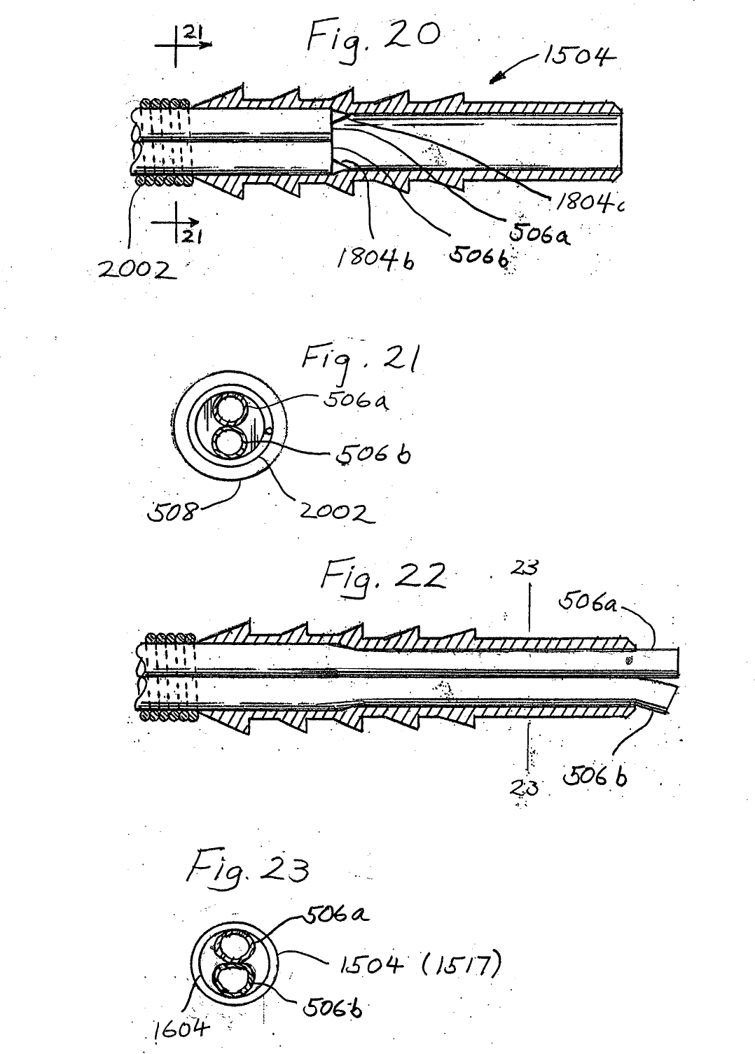

FIG. 24 is an isometric view and FIG. 25 is a top view of a chest piece 502 having improved acoustic properties. Chest piece 502 has an air passage 1507 open to sound collection chamber 2602. Unlike prior art chest pieces, air passage 1507 of FIGS. 24 and 25 enters sound collection chamber 2602 substantially straight. The lack of significant bends improves sound quality by eliminating obstructions, direction changes, and echoic surfaces that otherwise degrade the audio properties of prior art chest pieces. The sound collection chamber 2602 may be left open, or alternatively may be covered with a diaphragm, according to user preference.

When fitting 1502 is inserted into air passage 1507, wall 1806 blocks transmission of sound pressure except for that which is transmitted into passages 1802a and 1802b. Thus air passage 1507 forms a secondary sound collection chamber open to primary sound collection chamber 2602. When equipped with optional inner air tubes 506a and 506b, the seal provided by passages 1802a and 1802b in wall 1806 prevents leakage of sound pressure and improves audio volume at the ear tips 101.

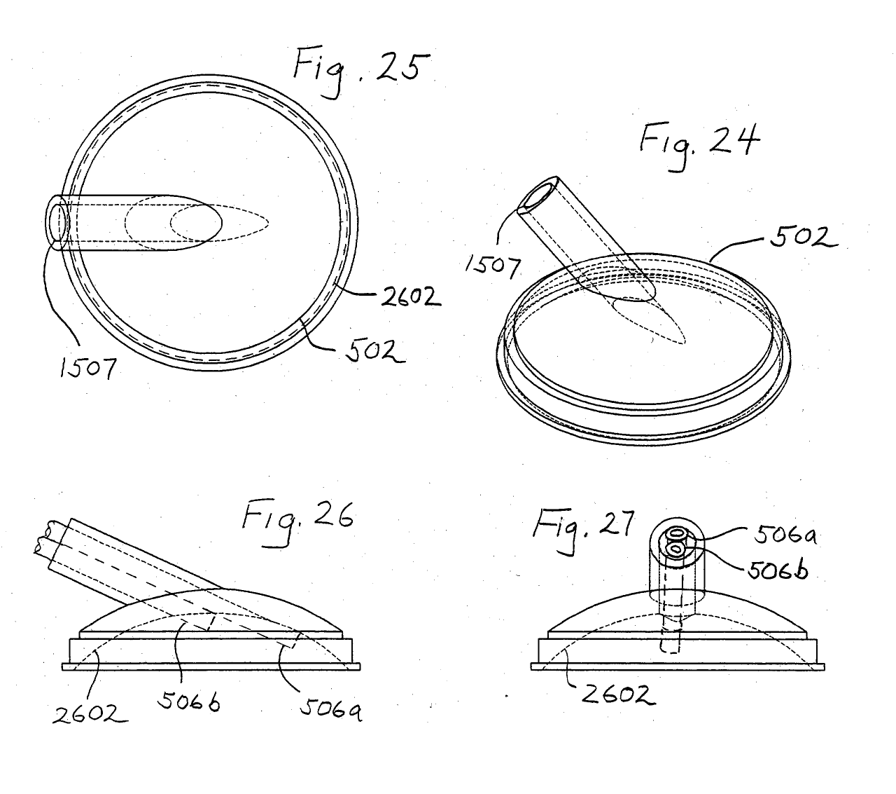

FIGS. 26 and 27 are side and rear views, respectively of the improved chest piece of FIGS. 25 and 26. These views show more clearly sound collection chamber 2602. Of note is the lack of parallel walls within the sound collection chamber. This helps to prevent standing waves from forming inside the sound collection chamber, also helping to improve sound quality by eliminating resonances. FIGS. 26 and 27 further illustrate an optional embodiment wherein inner air tubes 506a and 506b are passed through air passage 1507 to collect sound waves substantially directly from sound collection chamber 2602. This arrangement eliminates unwanted changes in the cross-sectional areas of the air columns carried by inner air tubes 506a and 506b as well as eliminating unwanted changes in the sound transmission qualities of the walls surrounding the air columns. This helps to improve sound volume, transient response and pass-band width at the ear tip. Keeping air passage 1507 straight or only subtly curving may be seen to have a special advantage when used with optional inner air tubes 506a and 506b, such gradual or no changes in direction thus preventing kinking of the inner air tubes.

FIGS. 26 and 27 additionally illustrate an additional alternative embodiment wherein the sound collection points of inner air tubes 506a and 506b are set at selected points across the diameter of sound collection chamber 2602. It is noted that sound qualities, and notably the spectral characteristics of sound, vary according to where sound is detected within sound collection chambers. In some cases, auscultation of low frequencies is enhanced near the periphery of the sound collection chamber whereas auscultation of higher frequencies is enhanced when the sound waves are collected from near the center of the sound collection chamber. The geometry of sound collection chamber 2602 may also be adjusted to enhance one vs. another portion of the audio spectrum. In the example shown, the smaller volume at the periphery may tend to emphasize higher frequencies, thus negating or reversing the trend for lower frequencies to dominate at the periphery of the sound collection chamber.

A further effect may be noticed in the perception of stereo effects, such spatial separation of sounds being more noticeable when the air columns carried to the listener are of somewhat differing length, thus producing a subtle phase delay that the user's brain interprets as stereo separation.

The length of inner air tubes 506a and 506b may be selected at the factory or alternatively may be left user-adjustable, the user thus selecting length variation and sound detection points according to personal preference and/or medical specialty. The user may easily select the lengths and locations by cutting the end of one or both of the inner air tubes with a pair of scissors or the like. Another optional embodiment, not shown, is the provision of air tube clips inside sound collection chamber 2602 that allow the user to position inner air tubes 506a and 506b according to his or her preference.

As was described earlier, the inventor has discovered that venting the air column has a significant effect on sound quality, sound pressure or volume, and ambient noise rejection. Generally speaking, greater sound volume may be gained by moderately increasing venting, while ambient noise may be more effectively rejected by decreasing or eliminating venting. FIGS. 28, 29, and 30 are top, side, and rear views, respectively of an improved chest piece having air column venting. The chest piece 502 is comprised of a body 2801 that contains a sound collection chamber 2602. An optional diaphragm (not shown) can substantially seal the sound collection chamber. Body vent passages 2802a, 2802b, and 2802c form pathways for vent air to enter the sound collection chamber. Adjustment ring 2804 is rotatably affixed around chest piece body 2801. Adjustment ring vent holes 2806a, 2806b, and 2806c are formed in the adjustment ring at centers spacing substantially equal to the center-to-center distances between body vent passages 2802a, 2802b, and 2802c. When the adjustment ring 2804 is rotated, the user may select the number of holes that align between the body and ring, thus selecting the amount of venting. Detents (not shown) may be employed to aid in aligning ring and body holes. FIGS. 28-30 illustrate an example where two adjustment ring holes, 2806a and 2806b are aligned with two body holes, 2802b and 2802c, respectively, thus providing a medium amount of venting. Rotating the ring one step counterclockwise would reduce the venting by aligning only one ring hole 2806a with one body hole, 2802c, thus producing a small amount of venting. Rotating one more step counterclockwise would prevent any holes from aligning, producing no venting. Rotating adjustment ring 2804 clockwise one step from the alignment shown in FIGS. 28-30 would cause three sets of holes to align, body hole 2802a with ring hold 2806a, body hole 2802b with ring hole 2806b, and body hole 2802c with ring hole 2806c; thus producing a large amount of venting.

Adjustment ring 2808 further includes indicator window 2808 which progressively exposes an increasing amount of indicator pattern 2809, formed under adjustment ring 2808 on the side of chest piece body 2801, as the adjustment ring is rotated clockwise. The amount of indicator pattern 2809 exposed is thus proportional to the amount of chest piece venting. The combination of indicator window 2808 and indicator pattern 2809 thus form an indicator that informs the user of the chest piece vent setting.

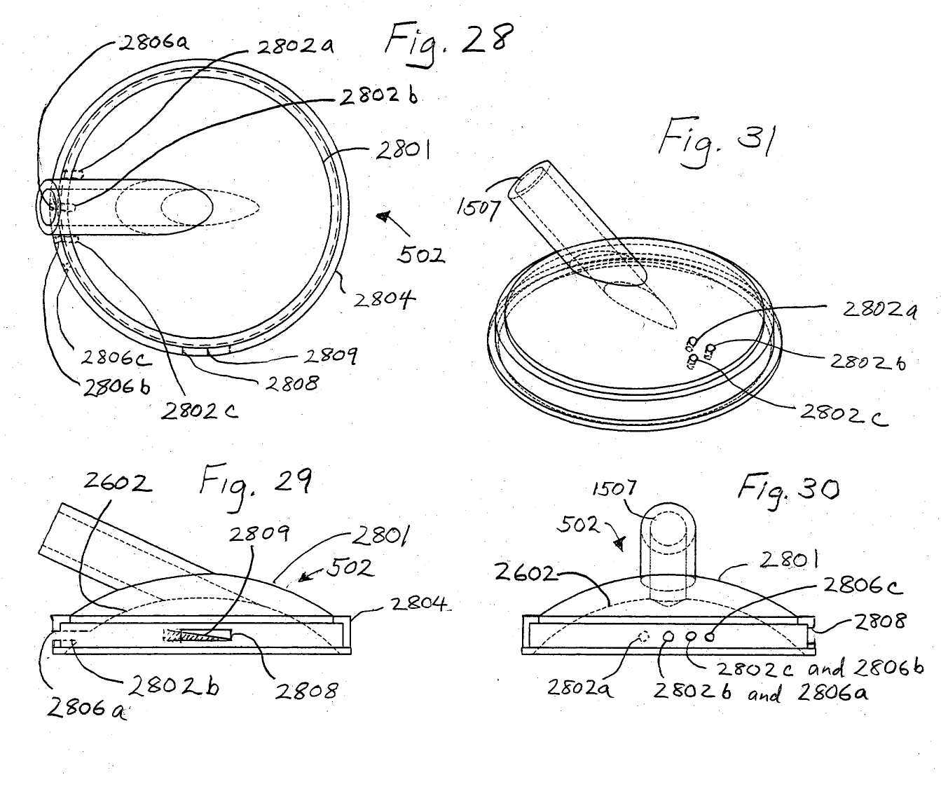

The example of FIGS. 28, 29, and 30 is one of a number of possible alternative embodiments. FIG. 31, for instance, illustrates an embodiment where the user may cover none, some, or all vent holes with his or her finger, an alternative embodiment that may be advantageous for dynamic environments with variable ambient noise and variable auscultation requirements. Many alternatives exist for venting into sound collection chamber 2602. Other alternative embodiments can vent into air passage 1507, fitting 1502, or through sheath 508, for instance.

FIGS. 32 through 35 illustrate the extension of inner air tubes into the sound collection chamber of the chest piece. FIG. 32 is a side view of horizontally-aligned inner air tubes 506 that are cut on a bias at the top of the sound collection chamber 2602. The bias cut 3202 and position at the top of the sound collection chamber has been found to improve acoustic efficiency and extend the frequency response of the system. FIG. 33 is a rear view of the chest piece configuration of FIG. 32.

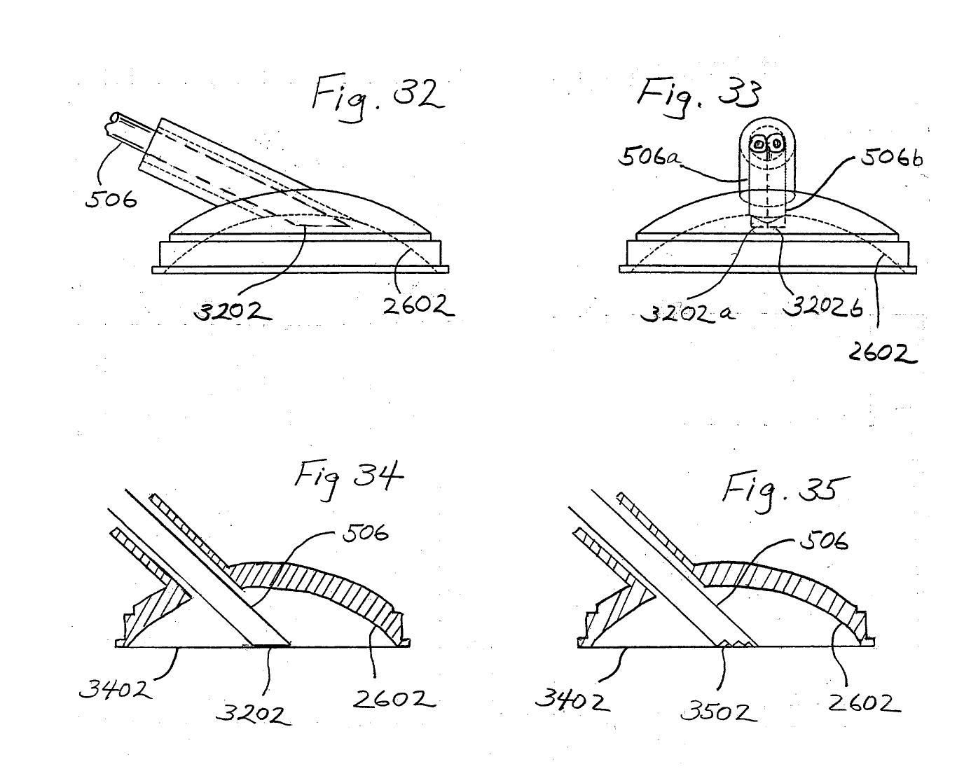

FIG. 34 is a sectional side view of a chest piece having air tubes 506 that are straight cut on a bias, the cut ends 3202 contacting diaphragm 3402. Extending the air tubes to contact the diaphragm in this way is especially advantageous for transmitting lower frequency portions of the auscultation signal. It is believed that the walls of the inner air tubes dampen higher frequency signals while passing lower frequency, higher energy signals.

FIG. 35 is a sectional side view of a chest piece having air tubes 506 that have serrated ends cut on a bias. The serration 3502 offers a compromise that boosts low frequency response while also allowing high frequency sound to pass. It is believed that the low frequency signal is contact-transmitted from the diaphragm through the tips of the serrations. Higher frequency signals pass from airborne sound waves in the sound collection chamber through the openings in the serrations.

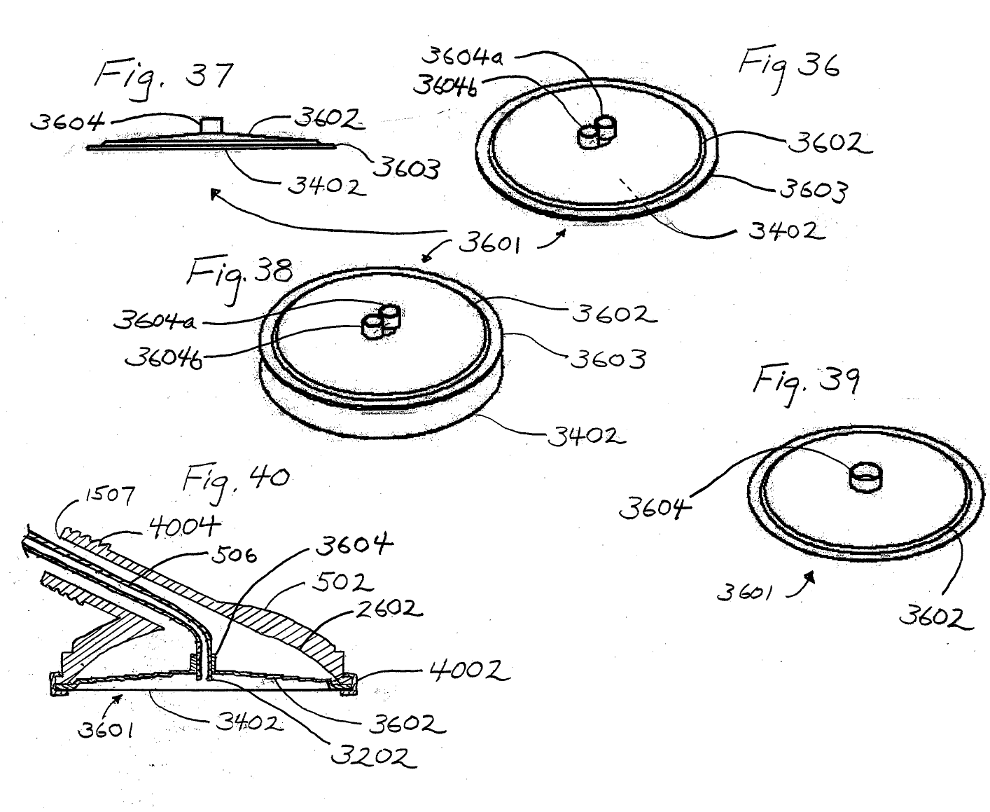

FIGS. 36 through 38 illustrate a binaural transducer cell 3601 that may be used to improve audio quality and/or isolation from ambient noise. FIG. 38 is an exploded view that draws attention to diaphragm 3402. FIG. 39 illustrates a monaural variant of the transducer cell. Both binaural and monaural variants are referred to as transducer cell 3601. FIG. 40 is a cross-sectional illustration of the transducer cell mounted in a chest piece 502.

Transducer top 3602 is formed in a generally conical shape and has a pair of air passages 3604a and 3604b extending therethrough. Transducer top 3602 may be formed of any appropriate material and preferably of an injection molded, thermoformed, or blow-molded plastic. Rim 3603 is circumferentially formed around transducer top 3602 and forms a surface for seating diaphragm 3402. Diaphragm 3402 may be formed of a metal or plastic membrane. It has been found that forming the diaphragm of PET or PVC in a thickness of 0.003 inches to 0.010 inches works well. Diaphragm 3402 may be permanently or removably attached to rim 3603 by clamping, gluing, heat welding, sonic welding, or other convenient method. In a preferred embodiment, diaphragm 3402 is sonically welded to rim 3603. In a preferred embodiment, diaphragm 3402 is hermetically sealed to rim 3603 forming an enclosed airspace within.

Transducer cell 3601 may be fitted to a chest piece body 502 as illustrated by FIG. 40. Rim 3603 seats against the rim of chest piece body 502 and is clamped thereto by retaining ring 4002. In an alternative embodiment, transducer cell 3601 may have substantially vertical walls extending from the periphery of rim 3603, such walls clamping around the periphery of chest piece body 502 and thus functionally replacing retaining ring 4002. Alternative attachment means will be clear to those skilled in the art. Inner air tubes 506a and 506b pass through air passages 3604a and 3604b, respectively, in the case of a binaural transducer. Alternatively, a single inner air tube 506 passes through air passage 3604 in the case of a monaural transducer. In either case, air passages 3604 form a tight seal around inner air tubes 506, thus ensuring substantially all sound pressure generated by the diaphragm is transmitted up through inner air tubes 506. In the example of FIG. 40, inner air tube ends 3202 are cut at a right angle at some distance from diaphragm 3402. Alternatively, ends 3202 may be angled and/or serrated, and formed either contacting or non-contacting with diaphragm 3402, as illustrated by FIGS. 32-35.

FIG. 40 also illustrates an integral nipple 4004 with flanges for seating an outer sheath 508 (not shown).

In the manner described, sound transducer 3601 forms a sealed sound collection chamber that is continuous with inner air tubes 506. In the embodiment of FIG. 40, an outer air chamber is formed between transducer top 3602 and chamber top 2602. This outer air chamber is advantageous for forming additional isolation from ambient noise and also may form an air suspension enclosure for transducer 3601. In embodiments where chest piece body 502 is selectably vented, the resonance frequency range of cone 3602, and thereby the resonance frequency range of transducer 3601, may be adjusted by selecting from a sealed air suspension environment or a ported environment as is known in the art of loudspeaker design. In a preferred embodiment, sound transducer 3601 is resonant at approximately 20-200 Hz. In another preferred embodiment, sound transducer 3601 is resonant at approximately 40-150 Hz and especially at 45-50 Hz. In this way, sound transducer 3601 may be advantageous for auscultation of the 45-50 Hz sounds that are both pathologically important and very subtle.

Transducer 3601 may form a replaceable transducing cell that is convenient for the user through elimination of the need to deal with seating fussy replacement diaphragms. Various models of transducers 3601 may be offered to optimize the stethoscope for various medical and healthcare disciplines or user preferences where a variety of resonance frequency ranges may be desirable.

Transducer 3601 may additionally add value to stethoscopes that do not have continuous inner air tubes 506. In this case a short length of inner air tube 506 may be threaded through chest piece air passage 1507 and coupled to outer air tubes 504 (not shown), air passages 1802 (not shown), or other acoustic connection.

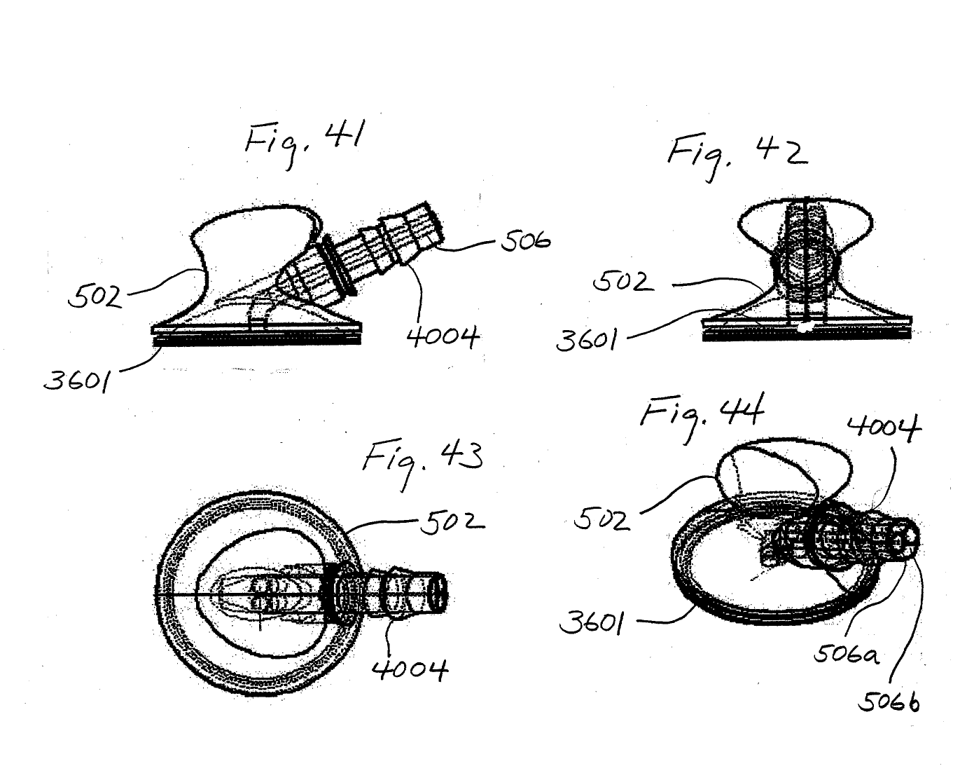

FIGS. 41 through 44 illustrate transducer 3601 mounted in alternative chest piece body 502 corresponding to the design of FIG. 12. In this example, chest piece body 502 includes integral nipple 4004 for attachment to sheath 508 (not shown). FIGS. 41 through 44 are further exemplary of a binaural transducer 3601 having two inner air tubes 506a and 506b extending therefrom.

The preceding overview of the invention, brief description of the drawings, and detailed description describe exemplary embodiments of the present invention in a manner intended to foster ease of understanding by the reader. Other structures, methods, and equivalents may be within the scope of the invention. As such, the scope of the invention described herein shall be limited only by the claims.

What is claimed is:

1. A stethoscope, comprising;
at least one chest piece having a sound collection chamber and an air passage acoustically coupled to said sound collection chamber for communicating sound pressure out of said chest piece,
at least one air tube acoustically coupled to the air passage of said chest piece,
a pair of binaurals acoustically coupled to said at least one air tube,
a binaural spring mechanically coupled to said pair of binaurals, and
a visual indicator formed on said stethoscope,
wherein said indicator indicates a proper orientation for use,
whereby a user can easily determine proper stethoscope orientation.

2. The stethoscope of claim 1, further comprising;
an indicator that indicates selected rotation angle of each of said pair of binaurals relative to said binaural spring.

3. A stethoscope, comprising;
a chest piece having a sound collection chamber,
a fitting coupled to said chest piece, said fitting providing a passage for communicating sound pressure from said chest piece,
a pair of outer air tubes coupled to said fitting, said pair of outer air tubes providing a passage for communicating sound pressure from said fitting,
a pair of binaurals coupled to said outer air tubes, said pair of binaurals providing a passage for communicating sound pressure from said outer air tubes, and
a pair of ear tips coupled to said pair of binaurals, said pair of ear tips providing a passage for communicating sound pressure from said outer air tubes,
wherein said fitting comprises;
an air chamber adjacent to said chest piece sound collection chamber, a pair of air passages, separated by a septum, in pneumatic communication with said air chamber, and
a tapered surface that provides a transition from said air chamber to said pair of air passages.

4. The stethoscope of claim 3, further comprising;
a pair of inner air tubes that provide a pair of passages for communicating sound pressure from said sound collection chamber to said pair of ear tips.

5. The stethoscope of claim 4, wherein said pair of inner air tubes are comprised of a polymeric material.

6. The stethoscope of claim 5, wherein said polymeric material is urethane.

7. The stethoscope of claim 4, wherein said pair of inner air tubes pass through said pair of air passages in said fitting, said pair of air passages in said fitting forming tight seals against the outside of said pair of inner air tubes.

8. The stethoscope of claim 4, wherein said pair of inner air tubes extend to and collect sound directly from said sound collection chamber.

9. The stethoscope of claim 8, wherein said pair of inner air tubes are cut on a bias.

10. The stethoscope of claim 8, wherein the ends of said pair of inner air tubes are extended to be in contact with a diaphragm.

11. The stethoscope of claim 8, wherein the walls of said pair of inner air tubes are perforated.

12. The stethoscope of claim 8, wherein the ends of said pair of inner air tubes are cut with an irregular edge.

13. The stethoscope of claim 8, wherein said pair of inner air tubes collect sound from separate locations within said sound collection chamber.

14. The stethoscope of claim 8, wherein said pair of inner air tubes are different lengths.

15. The stethoscope of claim 8, further comprising;
a package for containing said stethoscope, and
user directions contained by said package,
wherein said user directions contain instructions for cutting said inner air tubes to length.

16. The stethoscope of claim 4, wherein said pair of inner air tubes extend beyond the ends of said pair of binaurals, the ends of said inner air tubes being supported by said pair of ear tips.

17. The stethoscope of claim 16, wherein said ear tips further comprise exit bores, said exit bores being substantially equal in diameter to the inner diameters of said inner air tubes.

18. An earpiece for transmitting sound to a user from a pneumatic audio system, comprising;
a cylindrical body having a distal and a proximal end,
a plurality of flanges formed radially around said body, and
an air passage for carrying an air column formed within said body, said air passage extending longitudinally along the center of said body from the distal end to the proximal end,
wherein said plurality of flanges are formed with progressively increasing diameter from the proximal toward the distal end of said body.

19. The earpiece of claim 18, wherein the proximal end of said body may be trimmed to make a larger diameter flange the most proximal of flanges.

20. The earpiece of claim 18, wherein one or more flanges may be trimmed to provide venting of the air column to the ambient environment.

21. The earpiece of claim 18, further comprising one or more vent passages for venting the air column to the ambient environment.

22. The earpiece of claim 21, wherein the amount of said venting is adjustable.

23. The earpiece of claim 22, further comprising a visual indicator of the amount of venting selected.

24. The earpiece of claim 18, wherein said flanges are silicone.

25. The earpiece of claim 18,
wherein said body and said flanges are formed of a fluorescent color,
whereby a user may rapidly identify a personal device.

26. The earpiece of claim 18, wherein said air passage comprises;
a binaural bore for accepting a binaural, and
an inner air tube bore for accepting an inner air tube extending beyond the end of said binaural.

27. The earpiece of claim 18, wherein said pneumatic audio system is a stethoscope.

28. A stethoscope, comprising;
at least one chest piece having a sound collection chamber and an air passage carrying an air column for communicating sound pressure from said sound collection chamber,
at least one air tube carrying an air column acoustically coupled to the air passage of said at least one chest piece,
at least one binaural carrying an air column acoustically coupled to said at least one air tube,
an ear tip affixed to the end of said at least one binaural, and
at least one vent passage formed between the air column and the ambient environment.

29. The stethoscope of claim 28, wherein said at least one vent passage is formed in said ear tip.

30. The stethoscope of claim 29, wherein said vent passage has an adjustable amount of venting.

31. The stethoscope of claim 30, wherein said vent passage is adjusted by rotating said ear tip on said binaural.

32. The stethoscope of claim 30, further comprising;
a visual indicator for indicating the amount of venting.

33. The stethoscope of claim 28, wherein said sound collection chamber comprises a sealed transducer held by said chestpiece, said sealed transducer further comprising;
a transducer top having air passages formed therethrough through which pass said inner air tubes, and
a diaphragm hermetically sealed to said transducer top to enclose the sound collection chamber.

34. The stethoscope of claim 33, wherein said transducer top and the body of said chestpiece form an airspace therebetween.

35. The stethoscope of claim 34, wherein said airspace may be selectably vented.

36. The stethoscope of claim 34, wherein s aid sealed transducer has a resonance frequency range encompassing 20 Hz to 200 Hz.

37. The stethoscope of claim 36, wherein said sealed transducer has a resonance frequency range encompassing 45 Hz to 50 Hz.

38. The stethoscope of claim 33, wherein said transducer is replaceable.

39. The stethoscope of claim 28, wherein said vent passage is formed in said at least one chest piece.

40. The stethoscope of claim 39, wherein said vent passage has an adjustable amount of venting.

41. The stethoscope of claim 40, further comprising;
an indicator on said chest piece for indicating the amount of venting.

42. The stethoscope of claim 28, wherein said at least one chest piece consists of two chest pieces,
said at least one air tube consists of two air tubes, each of said two air tubes being acoustically coupled to one of said two chest pieces,
said at least one binaural consists of two binaurals, each of said two binaurals being acoustically coupled to one of said two air tubes.

43. A sound collector, comprising;
a body with a sound collection chamber formed therein, and
at least one air tube extending into said sound collection chamber.

44. The sound collector of claim 43, wherein the end of said air tube is cut at an angle not equal to 90 degrees.

45. The sound collector of claim 43, further comprising;
a diaphragm covering said sound collection chamber,
wherein the end of said air tube contacts said diaphragm.

46. The sound collector of claim 43, wherein the end of said air tube is serrated.

47. The sound collector of claim 43, wherein said sound collector is a sealed transducer.
